(19) United States
(12) Patent Application Publication
(10) Pub. No.: US 2023/0201596 A1
(43) Pub. Date: Jun. 29, 2023

(54) SYSTEMS AND METHODS FOR PERIPHERAL NEUROMODULATION

(71) Applicant: Boston Scientific Neuromodulation Corporation, Valencia, CA (US)

(72) Inventors: Tianhe Zhang, Studio City, CA (US); Rosana Esteller, Santa Clarita, CA (US)

(21) Appl. No.: 18/086,148

(22) Filed: Dec. 21, 2022

Related U.S. Application Data
(60) Provisional application No. 63/293,396, filed on Dec. 23, 2021.

(51) Int. Cl. A61N 1/36

(52) U.S. Cl. CPC A61N 1/36132; A61N 1/36135; A61N 1/36171; A61N 1/3615

(57) ABSTRACT

A system may include neuromodulation electrode contacts, a waveform generator, and a controller. The neuromodulation electrode contacts may be configured and arranged for use in delivering neuromodulation to a target peripheral nerve, where the target peripheral nerve includes a plurality of fibers, and the plurality of neuromodulation electrode contacts is configurable into a plurality of electrode configurations for stimulating different subsets of fibers within the plurality of fibers. The waveform generator may be configured for use to generate neuromodulation energy. The controller may be configured for use for identifying an electrode configuration that, when used to deliver the neuromodulation, stimulates fibers from an inhibitory surround receptive field, identifying a threshold amplitude corresponding to either a perception threshold or an evoked neural threshold for the identified electrode configuration, and delivering sub-perception therapy for the identified electrode configuration using a therapeutic amplitude that is set based on the threshold amplitude.

### CLAIM OF PRIORITY

This application claims the benefit of U.S. Provisional Application No. 63/293,396, filed on Dec. 23, 2021, which is hereby incorporated by reference in its entirety.

### TECHNICAL FIELD

This document relates generally to medical systems, and more particularly, but not by way of limitation, to systems, devices, and methods for providing peripheral neuromodulation.

### BACKGROUND

Therapy devices are devices configured to deliver a therapy. These devices may be external or implantable. Examples of therapy devices include electrical therapy devices such as neuromodulators and cardiac rhythm management devices, mechanical therapy devices, thermal therapy devices, and drug delivery devices. Examples of neuromodulators include, but are not limited to, spinal cord stimulators (SCS), deep brain stimulators (DBS), peripheral nerve stimulation (PNS) and function electrical stimulation (FES). Examples of cardiac rhythm management device include, but are not limited to, pacemakers and defibrillators. Examples of mechanical devices include, but are not limited to, devices configured to deliver compression to prevent deep vein thrombosis or to massage fluid from legs. Examples of drug delivery devices include, but are not limited to, insulin pumps or other infusion pumps.

With respect to neuromodulators, for example, an external programming device may be used to program the implantable neurostimulator with modulation parameters controlling the delivery of the neuromodulation energy. For example, modulation parameters may comprise electrode combinations, which define the electrodes that are activated as anodes (positive), cathodes (negative), and turned off (zero), percentage of modulation energy assigned to each electrode (fractionalized electrode configurations), and electrical pulse parameters, which define the pulse amplitude (measured in milliamps or volts depending on whether the pulse generator supplies constant current or constant voltage to the electrode array), pulse width (measured in microseconds), pulse rate (measured in pulses per second), and burst rate (measured as the modulation on duration X and modulation off duration Y).

Conventionally, the customization of values for these parameters to a patient can be very time costly. For example, the modulation parameters may be configured as a neuromodulation program capable of being implemented by the neuromodulator, and the neurostimulator may be programmed with more than one program. In order to find a program that provides an effectively provides a therapy (e.g., pain relief) with negligible side effects, the patient or clinician may implement different programs within the neuromodulator.

There is a need to improve the delivered neuromodulation, to be less invasive, and to enable faster programming.

### SUMMARY

An example (e.g., “Example 1”) of a system may include a plurality of neuromodulation electrode contacts, a waveform generator, and a controller. The plurality of neuromodulation electrode contacts may be configured and arranged for use in delivering neuromodulation to a target peripheral nerve, where the target peripheral nerve includes a plurality of fibers, and the plurality of neuromodulation electrode contacts is configurable into a plurality of electrode configurations for stimulating different subsets of fibers within the plurality of fibers. The waveform generator may be configured for use to generate neuromodulation energy. The controller may be configured for use for identifying an electrode configuration that, when used to deliver the neuromodulation, stimulates fibers from an inhibitory surround receptive field, identifying a threshold amplitude corresponding to a perception threshold, a motor or an electromyogram (EMG) threshold, or an evoked neural threshold for the identified electrode configuration, and delivering sub-perception therapy for the identified electrode configuration using a therapeutic amplitude that is set based on the threshold amplitude.

In Example 2, the subject matter of Example 1 may optionally be configured such that the controller is configured to identify the electrode configuration by delivering neuromodulation energy in a process to identify a neuromodulation configuration that stimulates the fibers from the inhibitory surround receptive field around the localized pain region. The identified neuromodulation configuration may include waveform parameters and the identified electrode configuration.

In Example 3, the subject matter of Example 2 may optionally be configured such that the waveform generator includes multiple independent current control (MICC) to provide independent current control to each of the neuromodulation electrode contacts. The identified neuromodulation configuration may include fractionalized current contributions to individual electrode contacts within the identified neuromodulation electrode configuration.

In Example 4, the subject matter of any one or more of Examples 1-3 may optionally be configured such that the controller is configured to identify the threshold amplitude by performing a threshold process. The threshold process may include: stepping up an adjustable amplitude of the neuromodulation until a patient perceives paresthesia; stepping up the adjustable amplitude until a neural response is evoked or suppressed; stepping up the adjustable amplitude until a muscle twitch or an EMG signal is evoked or suppressed; stepping down the adjustable amplitude of the neuromodulation until the patient fails to perceive the paresthesia; stepping down the adjustable amplitude until the neural response is evoked or suppressed; or stepping down the adjustable amplitude until the muscle twitch or the EMG signal is evoked or suppressed.

In Example 5, the subject matter of any one or more of Examples 1˜4 may optionally be configured to further include sensing electrode contacts that are configurable into a plurality of sensing configurations for sensing evoked neural responses in different subsets of fibers within the plurality of fibers, a data recorder configured to record data corresponding to the received electrical signal, a user input, and a display. The controller may be configured for use in identifying the electrode configurations by: using sensing electrode contacts to sense evoked neural responses; recording data corresponding to the received electrical signal; receiving a sensory input from a user via the user input; and display the sensed evoked responses and the received sensory input on a user interface.

In Example 6, the subject matter of Example 5 may optionally be configured such that the received sensory input from the patient includes at least one of pain, patient sensation or patient rating corresponding to test neuromodulation configurations.

In Example 7, the subject matter of Example 6 may optionally be configured such that the controller is configured for displaying the electrode configuration corresponding to the test neuromodulation configuration.

In Example 8, the subject matter of any one or more of Examples 1-7 may optionally be configured such that the controller is configured for use to identify the electrode configuration by: applying neuromodulation; receiving patient input regarding paresthesia or a motor or EMG response, and regarding pain; receiving input regarding evoked neural response in fibers of the peripheral nerve; and implementing an algorithm to distinguish between stimulation of fibers from the center receptive field or the inhibitory surround receptive field based on a relation among pain, and the evoked neural response including features in the evoked neural response, the paresthesia or the motor or EMG response.

In Example 9, the subject matter of Example 8 may optionally be configured such that the algorithm is configured to infer from an overlap in the patient sensation and the pain and from features the evoked neural response that low-threshold fibers from the center receptive field or the inhibitory surround receptive field is stimulated.

In Example 10, the subject matter of any one or more of Examples 8-9 may optionally be configured such that the algorithm is configured to infer from slight discordance among the patient sensation and the pain, and from slight distinctions in the evoked neural response that the fibers from the inhibitory surround receptive field is stimulated.

In Example 11, the subject matter of any one or more of Examples 8-10 may optionally be configured such that the algorithm is configured to infer from at least one of a strong sensation or a strong distinction in the evoked neural response that the inhibitory surround receptive field or a different receptive field is stimulated.

In Example 12, the subject matter of any one or more of Examples 1-11 may optionally be configured such that the therapeutic amplitude is less than the threshold amplitude and is set as a percentage of the threshold amplitude, and the neuromodulation configuration includes a pulse frequency of 90 Hz and a pulse width within a range of 210-230 μs.

In Example 13, the subject matter of any one or more of Examples 1-12 may optionally be configured such that the plurality of neuromodulation electrode contacts is on at least one cuff. an anchored patch, or a needle array.

In Example 14, the subject matter of any one or more of Examples 1-12 may optionally be configured to include an implantable device housing configured to house the waveform generator and the controller, where at least some of the plurality of neuromodulation electrode contacts are on the implantable device housing.

In Example 15, the subject matter of any one or more of Examples 1-12 may optionally be configured such that wherein the sensing electrode contacts are on a cuff for surrounding the peripheral nerve, configured to be implanted near at least one branch of the peripheral nerve, or configured to be externally worn.

Example 16 includes subject matter (such as a method, means for performing acts, machine readable medium including instructions that when performed by a machine cause the machine to perform acts, or an apparatus to perform). The subject matter may be performed using a plurality of neuromodulation electrode contacts configured and arranged for use in delivering neuromodulation to a target peripheral nerve, where the target peripheral nerve includes a plurality of fibers, and the plurality of neuromodulation electrode contacts is configurable into a plurality of electrode configurations for stimulating different subsets of fibers within the plurality of fibers. The subject matter may include: identifying an electrode configuration that, when used to deliver the neuromodulation, stimulates fibers from an inhibitory surround receptive field; identifying a threshold amplitude corresponding to a perception threshold, a motor or an EMG threshold, or an evoked neural threshold for the identified electrode configuration; and delivering sub-perception therapy for the identified electrode configuration using a therapeutic amplitude that is set based on the threshold amplitude.

In Example 17, the subject matter of Example 16 may optionally be configured such that the identifying the electrode configuration includes delivering neuromodulation energy in a process to identify a neuromodulation configuration that stimulates the fibers from the inhibitory surround receptive field around the localized pain region, where the identified neuromodulation configuration includes: waveform parameters, and the identified electrode configuration.

In Example 18, the subject matter of Example 17 may optionally be configured to further include independently controlling the current to each of the plurality of neuromodulation electrode contacts to control fractionalized current contributions to individual electrode contacts within the identified electrode configuration.

In Example 19, the subject matter of any one or more of Examples 16-18 may optionally be configured such that the identifying a threshold amplitude includes performing a threshold process, which may include stepping up an adjustable amplitude of the neuromodulation until a patient perceives paresthesia, stepping up the adjustable amplitude until a neural response is evoked or suppressed, stepping up the adjustable amplitude until a muscle twitch or an EMG signal is evoked or suppressed, stepping down the adjustable amplitude of the neuromodulation until the patient fails to perceive the paresthesia, stepping down the adjustable amplitude until the neural response is evoked or suppressed, or stepping down the adjustable amplitude until a muscle twitch or an EMG signal is evoked or suppressed.

In Example 20, the subject matter of any one or more of Examples 16-19 may optionally be configured such that the identifying the electrode configuration includes: using sensing electrode contacts to sense evoked neural responses, where the sensing electrode contacts are configurable into a plurality of sensing configurations for sensing evoked neural responses in different subsets of fibers within the plurality of fibers, and recording data corresponding to the received electrical signal.

In Example 21, the subject matter of Example 20 may optionally be configured such that the sensing electrode contacts are configured for sensing evoked neural responses on different sides of the peripheral nerve or distinct branches of the peripheral nerve. The subject may further include receiving a sensory input from a patient, and displaying the sensed evoked responses and the received sensory input on a user interface.

In Example 22, the subject matter of Example 21 may optionally be configured such that the received sensory input from the patient includes at least one of pain, patient sensation or patient rating corresponding to test neuromodulation configurations.

In Example 23, the subject matter of Example 22 may optionally be configured to further include displaying the electrode configuration corresponding to the test neuromodulation configurations.

In Example 24, the subject matter of any one or more of Examples 16-23 may optionally be configured such that the identifying the electrode configuration includes: applying super-perception neuromodulation; receiving patient input regarding paresthesia and pain; receiving input regarding evoked neural response in fibers of the peripheral nerve; and implementing an algorithm to distinguish between stimulation of fibers from the center receptive field or the inhibitory surround receptive field based on a relation among patient sensation, pain and the evoked neural response including features in the evoked neural response.

In Example 25, the subject matter of Example 22 may optionally be configured such that the algorithm is configured to infer from an overlap in the patient sensation and the pain and from features the evoked neural response that low-threshold fibers from the center receptive field or the inhibitory surround receptive field is stimulated.

In Example 26, the subject matter of any one or more of Examples 24-25 may optionally be configured such that the algorithm is configured to infer from slight discordance among the patient sensation and the pain, and from slight distinctions in the evoked neural response that the fibers from the inhibitory surround receptive field is stimulated.

In Example 27, the subject matter of any one or more of Examples 24-26 may optionally be configured such that the algorithm is configured to infer from at least one of a strong sensation or a strong distinction in the evoked neural response that the inhibitory surround receptive field or the different receptive field is stimulated.

In Example 28, the subject matter of any one or more of Examples 16-27 may optionally be configured such that the therapeutic amplitude is less than the threshold amplitude and is set as a percentage of the threshold amplitude.

In Example 29, the subject matter of any one or more of Examples 16-28 may optionally be configured such that the neuromodulation configuration includes a pulse frequency within a range of 50 Hz to 100 Hz and a pulse width within a range of 210-230 μs.

In Example 30, the subject matter of Example 29 may optionally be configured such that the pulse frequency is 90 Hz.

In Example 31, the subject matter of any one or more of Examples 16-30 may optionally be configured such that the identifying the electrode configuration includes: applying neuromodulation; receiving patient input regarding a motor or electromyogram response or an evoked neural response in fibers of the peripheral nerve; and implementing an algorithm to distinguish between stimulation of fibers from the center receptive field or the inhibitory surround receptive field based the patient input and the evoked neural response.

Example 32 includes subject matter (such as a device, apparatus, or machine) that may include non-transitory machine-readable medium including instructions, which when executed by a machine, cause the machine to perform a method using a plurality of neuromodulation electrode contacts configured and arranged for use in delivering neuromodulation to a target peripheral nerve, where the target peripheral nerve includes a plurality of fibers, and the plurality of neuromodulation electrode contacts is configurable into a plurality of electrode configurations for stimulating different subsets of fibers within the plurality of fibers. The method may include identifying an electrode configuration that, when used to deliver the neuromodulation, stimulates fibers from an inhibitory surround receptive field, identifying a threshold amplitude corresponding to a perception threshold, a motor or an EMG threshold, or an evoked neural threshold for the identified electrode configuration, and delivering sub-perception therapy for the identified electrode configuration using a therapeutic amplitude that is set based on the threshold amplitude.

In Example 33, the subject matter of Example 32 may optionally be configured such that the identifying the electrode configuration includes delivering neuromodulation energy in a process to identify a neuromodulation configuration that stimulates the inhibitory surround receptive field around the localized pain region, wherein the identified neuromodulation configuration includes the identified electrode configuration from the plurality of electrode configurations.

In Example 34, the subject matter of any one or more of Examples 32-33 may optionally be configured such that the therapeutic amplitude is less than the threshold amplitude and is set as a percentage of the threshold amplitude.

In Example 35, the subject matter of any one or more of Examples 32-34 such that the neuromodulation configuration includes a pulse frequency of 90 Hz and a pulse width within a range of 210-230 μs.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, a portion of a spinal cord including white matter and gray matter of the spinal cord.

FIG. 2 illustrates an embodiment of a neuromodulation system.

FIG. 3 illustrates, by way of example and not limitation, an example of a neuromodulation system.

FIG. 4 illustrates an embodiment of a modulation device, such as may be implemented in the neuromodulation system of FIG. 2.

FIG. 5 illustrates an embodiment of a programming device, such as may be implemented as the programming device in the neuromodulation system of FIG. 2.

FIG. 6 illustrates, by way of example, a model for local excitatory and inhibitory surround receptive fields in the dorsal column.

FIG. 7 illustrates, by way of example and not limitation, an implantable device configured to use a lead to deliver peripheral neuromodulation.

FIG. 8 illustrates, by way of example and not limitation, an implanted device configured to deliver peripheral neuromodulation using electrodes on housing of the implanted device.

FIG. 9 illustrates, by way of example and not limitation, an implantable device configured to perform peripheral nerve stimulation and perform peripheral nerve sensing on separate nerve aspects.

FIG. 10 illustrates, by way of example and not limitation, an implantable device with at least a first cuff for use to provide neuromodulation and a second cuff for use to sense evoked potentials.

FIG. 11 is a simple illustration of different stimulation thresholds for different fiber types in a complex nerve.

FIG. 12 illustrates, by way of example and not limitation, stimulation or sensing electrodes around a peripheral nerve and the capability to either stimulate or sense neural activity in some fibers and not other fibers.

FIG. 13 illustrates, by way of example and not limitation, signals from different nerve branches which may be displayed on a graphical user interface.

FIG. 14 illustrates, by way of example and not limitation, signals from the same nerve branches which may be displayed on a graphical user interface.

FIG. 15 illustrates, by way of example and not limitation, some neuromodulation parameters that may contribute to a neuromodulation configuration.

FIG. 16 illustrates, by way of example and not limitation, a method for calibrating and delivering a sub-perception therapy using peripheral neuromodulation.

FIG. 17 illustrates, by way of example and not limitation, a method for calibrating the peripheral neuromodulation for surround receptive field(s) and/or center receptive field(s) using patient inputs and/or sensed evoked neural responses.

FIG. 18 illustrates, by way of example and not limitation, a general workflow for closed-loop control of a neuromodulation therapy.

FIG. 19 illustrates, by way of example and not limitation, programming configurations A and B for providing neuromodulation to two different areas.

FIG. 20 illustrates, by way of example and not limitation, a fully implantable PNS device and an external programmer.

FIG. 21 illustrates, by way of example and not limitation, a partially implantable PNS device.

FIG. 22 illustrates, by way of example and not limitation, a partially implantable PNS device.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

A brief description of the spinal cord and peripheral nerves is provided herein to assist the reader in understanding the present subject matter. Generally, the nervous system includes the central nervous system and the peripheral nervous system. The central nervous system includes the brain and spinal cord, and the peripheral nervous system includes the autonomic nervous system, which regulate involuntary physiologic processes, and the somatic nervous system. The somatic nervous system includes sensory and motor nerves. There are twelve pairs of cranial nerves and thirty-one pairs of spinal nerves in the peripheral nervous system. Somatosensory information is provided by afferent nerves within the peripheral nervous system to the spinal cord, and motor control information is provided from the spinal cord to efferent nerves within the peripheral nervous system.

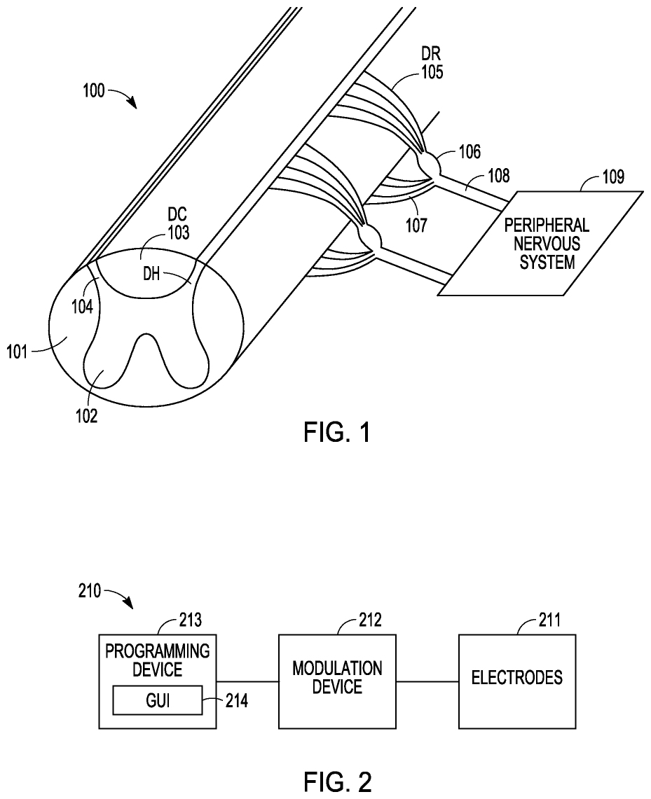

FIG. 1 illustrates, by way of example, a portion of a spinal cord 100 including white matter 101 and gray matter 102 of the spinal cord. The gray matter 102 includes cell bodies, synapse, dendrites, and axon terminals. Thus, synapses are located in the gray matter. White matter 101 includes myelinated axons that connect gray matter areas. A typical transverse section of the spinal cord includes a central “butterfly” shaped central area of gray matter 102 substantially surrounded by an ellipse-shaped outer area of white matter 101. The white matter of the dorsal column (DC) 103 includes mostly large myelinated axons that form afferent fibers that run in an axial direction. The dorsal portions of the “butterfly” shaped central area of gray matter are referred to as dorsal horns (DH) 104. In contrast to the DC fibers that run in an axial direction, DH fibers can be oriented in many directions, including perpendicular to the longitudinal axis of the spinal cord. Examples of spinal nerves are also illustrated, including a dorsal root (DR) 105, dorsal root ganglion (DRG) 106 and ventral root 107. The dorsal root 105 mostly carries sensory signals into the spinal cord. Cell bodies of the sensory neurons are in the DRG. The ventral root functions as an efferent motor root. Cell bodies of the motor neurons are in the gray matter. The dorsal and ventral roots join to form mixed spinal nerves 108 that provide motor, sensory and autonomic functions. The mixed spinal nerves form part of the peripheral neural system 109. Most spinal nerves pass through an intervertebral foramen and then divide into branches, some of which provide sensory information from various areas of the skin.

SCS has been used to alleviate pain. A therapeutic goal for conventional SCS programming has been to maximize stimulation (i.e., recruitment) of the DC fibers that run in the white matter along the longitudinal axis of the spinal cord and minimal stimulation of other fibers that run perpendicular to the longitudinal axis of the spinal cord (dorsal root fibers, predominantly), as illustrated in FIG. 1. The white matter of the DC includes mostly large myelinated axons that form afferent fibers. While the full mechanisms of pain relief are not well understood, it is believed that the perception of pain signals is inhibited via the gate control theory of pain, which suggests that enhanced activity of innocuous touch or pressure afferents via electrical stimulation creates interneuronal activity within the DH of the spinal cord that releases inhibitory neurotransmitters (Gamma-Aminobutyric Acid (GABA), glycine), which in turn, reduces the hypersensitivity of wide dynamic range (WDR) sensory neurons to noxious afferent input of pain signals traveling from the dorsal root (DR) neural fibers that innervate the pain region of the patient, as well as treating general WDR ectopy. Consequently, the large sensory afferents of the DC nerve fibers have been targeted for stimulation at an amplitude that provides pain relief. Current implantable neuromodulation systems used to provide SCS therapy typically include electrodes implanted adjacent, i.e., resting near, or upon the dura, to the dorsal column of the spinal cord of the patient and along a longitudinal axis of the spinal cord of the patient.

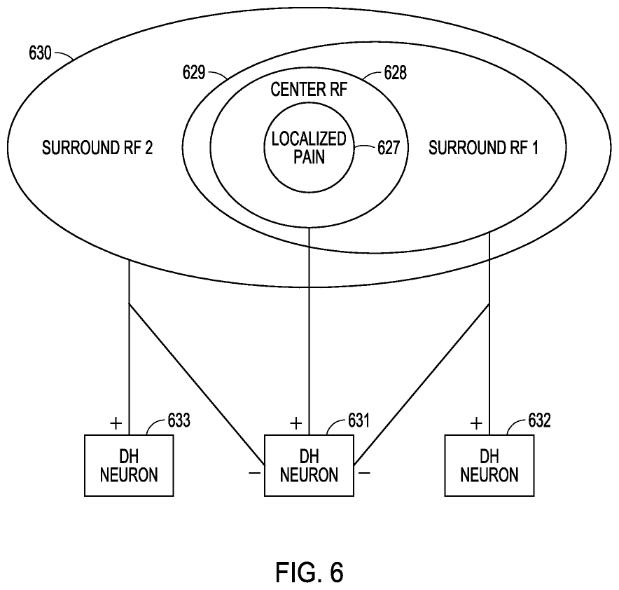

A sensory neuron responds, via electrical activity or action potentials, to a stimulation of a corresponding receptive field for the neuron. A receptive field includes sensory receptors that feed into the neuron as well as other receptors that use synaptic connection so to activate the neuron. Receptors synapse on neurons, and these neurons have center receptive fields that correspond to a region where the receptors directly innervate the neuron, and surround receptive fields that correspond to region(s) there the receptors indirectly communicate via inhibitory neurons with the neuron, as will be discussed in more detail with the model illustrated in FIG. 6. At a high-level concept, the central field indicates the location in which a stimulus must occur in order to cause a response from the sensory neuron. The surround receptive field provides a lateral inhibition for the center receptive field, enhancing stimulus perception near the edge of the center receptive fields.

Aspects of the present subject matter are directed toward providing surround inhibition for a localized pain region using peripheral nerve stimulation. It has been reported that activation of both local and surround receptive fields with respect to pain is required for SCS pain relief (Hillman and Wall, 1969; Zhang et al., 2014) and for proper configuration of patterns, waveforms, staggered stim if dorsal columns are a principal mechanism. Surround dorsal columns have their origins as peripheral afferent fibers that may innervate multiple levels of the spinal cord and can be accessed via peripheral nerve stimulation, as demonstrated by 1987 Smith and Bennet, where peripheral fibers were mapped to center and surround in the spinal cord.

FIG. 2 illustrates an embodiment of a neuromodulation system. The illustrated system 210 includes electrodes 211, a modulation device 212, and a programming device 213. The electrodes 211 are configured to be placed on or near one or more neural targets in a patient, such as one or more peripheral nerve targets. The modulation device 212 is configured to be electrically connected to electrodes 211 and deliver neuromodulation energy, such as in the form of electrical pulses or other waveform shape, to the one or more neural targets though electrodes 211. The modulation device 212 may be an implantable device or an external device with leads percutaneously inserted to be positioned to stimulate a peripheral nerve. The delivery of the neuromodulation is controlled by using a plurality of modulation parameters, such as modulation parameters specifying the electrical pulses and a selection of electrode(s) to function as anode(s) and a selection of electrode(s) to function as cathode(s) through which each of the electrical pulses is delivered. The modulation parameter may also include the fractional distribution of energy (e.g., current) provided across the anodic electrode(s) and cathodic electrode(s). In various embodiments, at least some parameters of the plurality of modulation parameters are programmable by a user, such as a physician or other caregiver. The programming device 213 provides the user with accessibility to the user-programmable parameters. In various embodiments, the programming device 213 is configured to be communicatively coupled to modulation device via a wired or wireless link. In various embodiments, the programming device 213 includes a graphical user interface (GUI) 214 that allows the user to set and/or adjust values of the user-programmable modulation parameters.

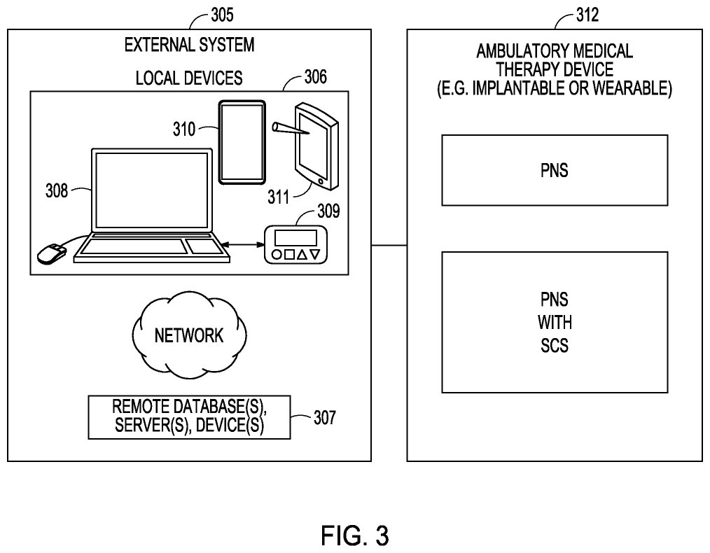

FIG. 3 illustrates, by way of example and not limitation, an example of a neuromodulation system. The illustrated neuromodulation system 311 includes an external system 312 that may include at least one programming device. The illustrated external system 312 may include device(s), at least some of which are configured for use by a clinician to communicate with and program a therapy device, such as an implantable neuromodulator. The external system 312 may include a network of computers, including local device(s) 313 and remote systems 314. The local device(s) 206 may include one or more of a programmer 315, a remote control 316, a phone 317 (e.g., smartphone), and a tablet 318. The remote system 314 may include remote database(s), remote server(s) and remote device(s) capable of communication through the network to local device(s). The external system 312 may be configured for use to program or receive data from the ambulatory therapy device 319 (e.g., a device configured to deliver therapy when the patient is ambulatory away from a clinical setting). The ambulatory therapy device may include at least one peripheral nerve stimulation (PNS) device. The PNS device may be implantable or external. The PNS device is generally less invasive than a spinal cord stimulation (SCS) device. In some embodiments, the system may include both a PNS device and a SCS device such as may be useful to provide a variety of therapy options to treat pain. For example, the clinical programmer 316 may be used to program the therapy device 319, using input from remote systems 314 or feedback received from other local devices 313. By way of example and not limitation, the remote control device 316 may allow the patient to turn a therapy on and off and/or may allow the patient to adjust patient-programmable parameter(s) of the plurality of modulation parameters, and the phone 317 or tablet 318 may be used to answer questionnaires to provide input for therapy control. The neuromodulation device may be an implantable device or an external device such as a wearable device. The external system 312 may include a wearable(s) such as a watch, sensors or therapy-applying devices. The watch may include sensor(s), such as sensor(s) for detecting activity, motion and/or posture. Other wearable sensor(s) may be configured for use to detect various physiological parameters such as, but not limited to, activity, motion and/or posture of the patient that may be useful input to control the therapy delivery.

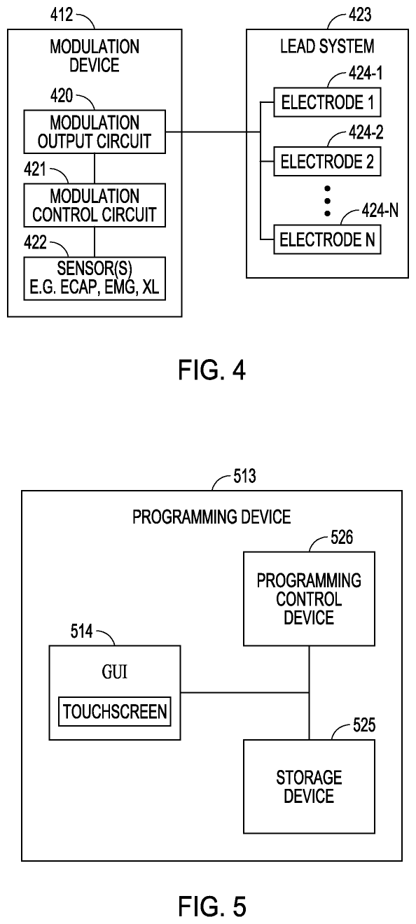

FIG. 4 illustrates an embodiment of a modulation device 412, such as may be implemented in the neuromodulation system 210 of FIG. 2. The modulation device 412 may be an implantable device or an external device with leads percutaneously inserted to be positioned to stimulate a peripheral nerve. The illustrated embodiment of the modulation device 412 includes a modulation output circuit 420 and a modulation control circuit 421. The modulation device 412 may include sensor(s) 422 for patient monitoring and/or feedback control of the therapy, telemetry circuitry and power. The modulation output circuit 420 may produce and deliver the neuromodulation waveform (e.g., neuromodulation pulses). The modulation control circuit 421 may control the delivery of the neuromodulation pulses using the plurality of modulation parameters. The lead system 423 includes one or more leads each configured to be electrically connected to modulation device 424 and a plurality of electrodes 424-1 to 424-N distributed in an electrode arrangement using the one or more leads, where N>2. Each lead may have an electrode array consisting of two or more electrodes, which also may be referred to as contacts. Multiple leads may provide multiple electrode arrays to provide the electrode arrangement. Each electrode is a single electrically conductive contact providing for an electrical interface between modulation output circuit 420 and tissue of the patient. The neuromodulation pulses are each delivered from the modulation output circuit 420 through a set of electrodes selected from the electrodes 424-1 to 424-N. The number of leads and the number of electrodes on each lead may depend on, for example, the distribution of target(s) of the neuromodulation and the need for controlling the distribution of electric field at each target. The neuromodulation system may be configured to therapeutically modulate the neural tissue. The therapeutic modulation may be supra-perception modulation or sub-perception modulation. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, pulse width, and rate (or frequency) of the electrical pulses. Each electrode configuration, along with the electrical pulse parameters, can be referred to as a “modulation parameter set.” Each set of modulation parameters, including fractionalized current distribution to the electrodes (as percentage cathodic current, percentage anodic current, or off), may be stored and combined into a modulation program that can then be used to modulate multiple regions within the patient.

FIG. 5 illustrates an embodiment of a programming device 513, such as may be implemented as the programming device 213 in the neuromodulation system of FIG. 2. The programming device 513 includes a storage device 525, a programming control circuit 526, and a GUI 514. The programming control device 526 generates the plurality of modulation parameters that controls the delivery of the neuromodulation pulses according to the pattern of the neuromodulation pulses. In various embodiments, the GUI 514 includes any type of presentation device, such as interactive or non-interactive screens, and any type of user input devices that allow the user to program the modulation parameters, such as touchscreen, keyboard, keypad, touchpad, trackball, joystick, and mouse. The storage device 525 may store, among other things, modulation parameters to be programmed into the modulation device. The modulation parameters may be organized into one or more sets of modulation parameters. The programming device 513 may transmit the plurality of modulation parameters to the modulation device. In some embodiments, the programming device 513 may transmit power to the modulation device. The programming control circuit 526 may generate the plurality of modulation parameters. In various embodiments, the programming control device 526 may check values of the plurality of modulation parameters against safety rules to limit these values within constraints of the safety rules.

In various embodiments, circuits of neuromodulation, including its various embodiments discussed in this document, may be implemented using a combination of hardware, software and firmware. For example, the GUI, modulation control, and programming control, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 6 illustrates, by way of example, a model for local excitatory and inhibitory surround receptive fields in the dorsal column. The model illustrates, for simplicity, an area of localized pain 627, a center received field 628, a surround receptive field 1 629, a surround receptive field 2 6230, and three dorsal horn (DH) neurons 631, 632 and 633. The center receptive field 628 has receptors that directly innervates to activate DH neuron 631. The surround receptive field 1 629 and surround receptive field 2 630 also have receptors that directly innervate to activate their corresponding DH neuron 632, 633, but they also indirectly communicate with and inhibit DH neuron 631. As provided above, a sensory neuron responds, via electrical activity or action potentials, to a stimulation of a corresponding receptive field for the neuron. A receptive field includes sensory receptors that feed into the neuron as well as other receptors that use synaptic connection so to activate the neuron. These neurons have center receptive fields that correspond to a region where the receptors directly innervate the neuron, and surround receptive fields that correspond to region(s) there the receptors indirectly communicate via inhibitory neurons with the neuron. The Center and Surround RFs send off dorsal column fibers that connect to dorsal horn (DH) neurons, which is where the +/−network connections happen. Neuromodulation devices, regardless of whether the devices are implantable devices or non-invasive external devices, are typically placed to activate the fibers originating from the receptive field, as opposed to the receptive field itself, which is the actual skin.

Aspects of the present subject matter are directed toward providing surround inhibition for a localized pain region using peripheral nerve stimulation. It has been reported that activation of both local and surround receptive fields with respect to pain is required for SCS pain relief (Hillman and Wall, 1969; Zhang et al., 2014) and for proper configuration of patterns, waveforms, staggered stim if dorsal columns are a principal mechanism. Surround dorsal columns have their origins as peripheral afferent fibers that may innervate multiple levels of the spinal cord and can be accessed via peripheral nerve stimulation, as demonstrated by 1987 Smith and Bennet, where peripheral fibers were mapped to center and surround in the spinal cord.

Various embodiments provide a system that enables the delivery and titration of arbitrary neuromodulation waveforms. The system may enable FAST to a peripheral nerve via one of several means. The device delivery package, including traditional implantable device, wirelessly controlled system, and a non-invasive system. System and method may detect the activation of center vs. surround receptive fields via e-physical recordings from neighboring nerves and/or the implant site. Closed loop control using applied stimulation may be based on acquired signals.

FAST neuromodulation provides fast-acting subperception therapy for a patient. FAST may include a fitting regime where a patient is tested with supra-perception stimulation (that the patient can feel; that produces paresthesia) to try and find a correct location for stimulation in their electrode array that well “covers” the patient's pain. Finding a correct location for stimulation is typically called “sweet spot searching,” because the goal is to find a “sweet spot” in the array for stimulation that well recruits and treats the patient's symptoms, such as lower back pain. Once this “sweet spot” for stimulation in the electrode array is located, the amplitude of the stimulation is lowered to provide sub-perception stimulation (that the patient can't feel), as explained further below.

Both the sweet spot searching the eventually-determined sub-perception stimulation therapy may use a low-frequency (e.g., 90 Hz) active recharge waveform. However, the frequency may be within a range between 1 Hz to 500, a range between 10 Hz and 200 Hz, a range between 50 Hz and 150 Hz, a range between 50 and 100, or a range between 80 and 100 Hz. The 90 Hz frequency is a specific example of a desirable parameter value. The pulse width may be 210 us, or within a range between 30 μs to 500 μs. The active recharge waveform is biphasic, because it includes two opposite-polarity phases that are both actively driven with constant currents of opposite polarity. Active recharge waveforms recover charge during the second pulse phase (recharge) that was injected during the first pulse phase. Specifically, when current is actively driven during the first pulse phase, charge will be stored on capacitances in the current path. When the polarity and hence direction of the current is reversed during the second phase, such stored charge is actively recovered and pulled off those capacitances. The active recharge waveform used during FAST is symmetric as the amplitude and duration of the two actively-driven pulse phases are the same. However, FAST may be designed to be implemented using asymmetrical pulses.

It is not conventional to use an active recharge waveform at low frequencies in an IPG. Rather, a passive recharge waveform, which includes only a first actively-driven first pulse such as a monophasic, cathodic pulse, is conventionally used as low frequencies. Rather than actively driving a current, passive charge recovery may involve connecting the electrodes to a common voltage causing any stored charge in the current paths to equilibrate by exponential decay through the patient's tissue. Passive recharge is more energy efficient than active recharge since a current is only actively drive during one phase.

A benefit of the active recharge waveform for FAST neuromodulation is that it effectively provides two center points of stimulation using a bipole. A first pole of the bipole may be a cathode pole during the first phase and an anode pole during a second phase, and the second pole of the bipole may be an anode pole during the first phase and a cathode pole during the second phase. These anode and cathode poles need not correspond to the exact positions of the electrodes in the array, but can instead be formed as “virtual poles” between the electrodes.

It is hypothesized that an active recharge waveform affects stimulation at these two CPS locations, which facilitates the identification and optimization of stimulation to patient-specific sweet-spot(s) for pain relief. As a result, when the amplitude of the stimulation is later dropped at this location to sub-perception levels, the source of pain remains well recruited, and provides the patient “FAST” relief from their symptoms. While still providing fast-acting symptomatic relieve, the low-frequency waveforms used in FAST use less power than sub-perception therapy delivered at higher frequencies (e.g., 10 kHz).

By way of example and not limitation, a FAST procedure may include trolling at a low intensity to cover the patient's worst painful area with paresthesia, then turn stimulation down to a percentage (e.g. 70%) of the perception threshold, assess pain including pain while performing an activity (e.g., walking), and if pain reduction is not excellent and very quick (e.g., under 5 minutes), then find a better sweet spot by continuing to troll to cover the painful area and turn stimulation down to the percentage until the pain reduction is excellent and very quick. Once excellent and very quick pain relief is achieved, then the perception threshold may be measured. The sub-perception therapy's maximum amplitude may be set at or otherwise based on the perception threshold. The program may be set to a percentage (e.g., 30%) lower than the perception threshold.

Additional information regarding FAST neuromodulation may be found in the following references, which are herein incorporated by reference in their entirety: U.S. Provisional application Ser. No. 17/347,348, U.S. Pat. No. 10,576,282, US Published App. No. 2020/0009367, 2020/0009394, 2020/0046980, 2020/0147397, 2020/0147390, 2020/0147392, 2020/0147393, 2020/0147388, 2020/0254256, 2020/0147400, and 2020/0147391, and PCT applications WO 2021/003290, WO 2021/0141652 and PCT/US2021/016867.

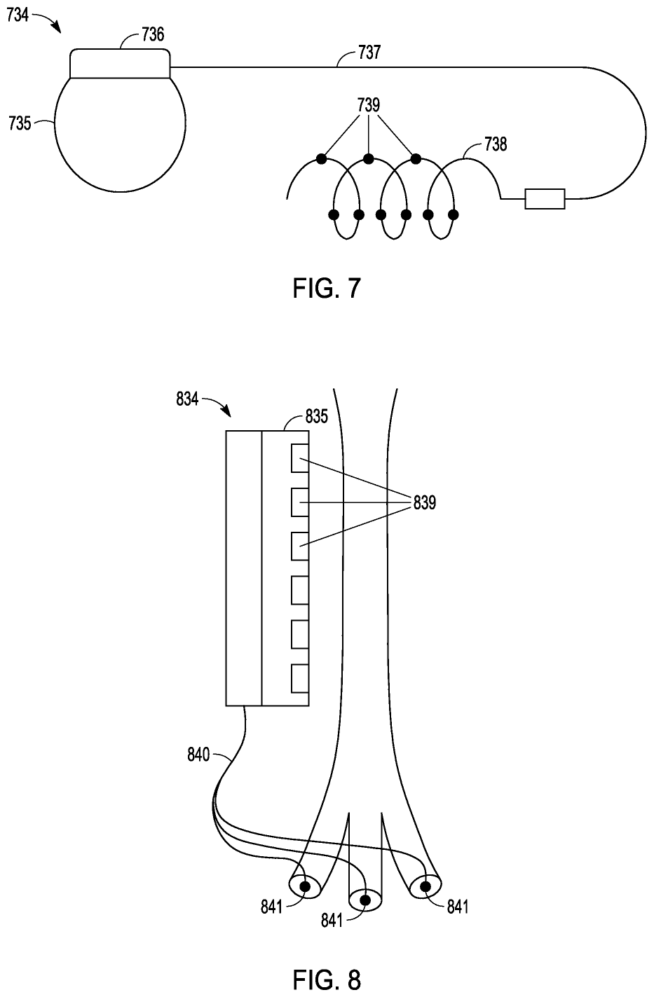

FIG. 7 illustrates, by way of example and not limitation, an implantable device configured to use a lead to deliver peripheral neuromodulation. The implantable device 734 may include a hermetically-sealed housing 735, also referred to as a “can”, with a header 736 used to connect to a proximal end of one or more leads 737. A battery and waveform generator may be contained within the housing. The distal end of the lead(s) 737 may be attached to a nerve cuff or a helical-shaped connector 738 configured to be secured around a target nerve. The nerve cuff or helical-shaped connector 738 may include a plurality of electrode contacts 739 that may be individually activated and controlled to create different modulation field shapes in the target nerve. For example, the electrode contacts may be programmable with MICC, and configurable with FAST waveform and anode/cathode pairing to try to capture surround inhibition. In other embodiments, the electrode contacts may be incorporated on anchored patch configured to be anchored at least partially around the nerve or near the nerve, or a needle array where each needle provides an electrode contact.

FIG. 8 illustrates, by way of example and not limitation, an implanted device 834 configured to deliver peripheral neuromodulation using electrodes 839 on housing 835 of the implanted device. The implantable device itself may be implanted onto/next to a target neurite. The hermetically-sealed housing 835 may include a header 836 used to connect to a proximal end of one or more leads 840 (e.g., sensing lead(s) with sensing contact(s) 841). These optional sensing contacts, which also may be referred to as recording electrodes, may be used to detect evoked potentials indicating single or multiple branch (surround) capture. A battery and waveform generator may be contained within the housing 835. The electrode contacts 839 on the housing 835 may be individually activated and controlled to create different modulation field shapes in the target nerve. The contacts may be configurable with FAST waveform and anode/cathode pairing to try to capture surround inhibition.

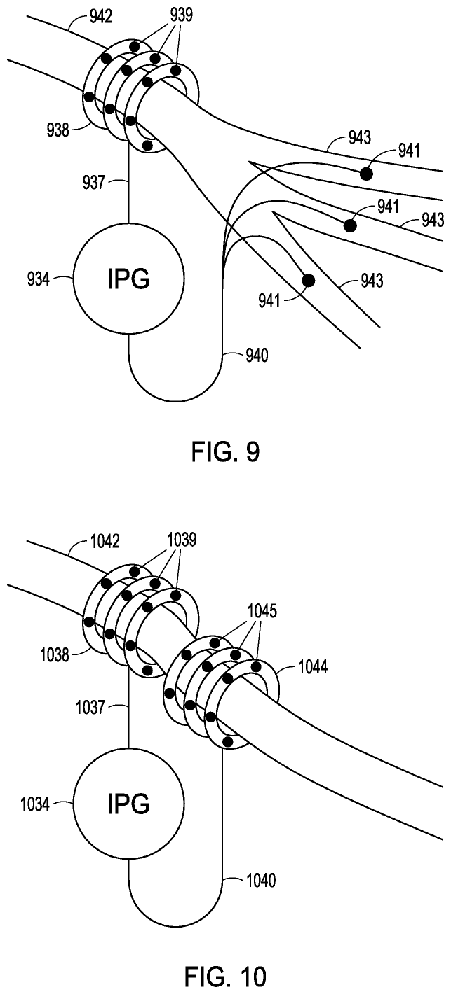

FIG. 9 illustrates, by way of example and not limitation, an implantable device 934 configured to perform peripheral nerve stimulation and perform peripheral nerve sensing on separate nerve aspects. In the illustrated embodiment, a lead 937 with a connected helical coil 938 may be used with electrodes 939 thereon for use to deliver neural stimulation to a nerve 942 or some fibers within the nerve. Individual ones of the stimulation contacts with specific fractionalizations of their respective amplitude may be selected to provide the modulation field over some of the fibers on the nerve but not others. Another lead may include sensing contacts to sense different nerve branches 943. Thus, a separate sensing lead 940 may be used to place sensing contacts 941 to sense neural activity on a distal branch and/or other nerve site where distinct groups of fibers can be collected. These distinct sensing contacts may be placed (usually) distal to the stimulation site, on e.g., separate nerve aspects. For example, sensing contacts may be place to sense neural activity on branches from different receptive fields (RFs), from different sides of the same nerve, or from different or particular dorsal root branches. It is noted that sensory nerves are afferent, as naturally generated nerve activity tends to originate at a peripheral nerve ending and propagate from the periphery to the brain (i.e., orthodromically) along those fibers. Neuromodulation may stimulate the nerves in the middle of the nerve rather than at the nerve ending. As such, the action potentials usually propagate both orthodromically and antidromically. The antidromic conduction may be sensed distally (e.g., distal from the stimulation site, which is further to periphery in this case) following an artificial stimulation pulse. Sensory nerves from different receptive fields tend to merge as they move towards the nervous system (e.g., the tibial/saphenous/peroneal nerves merge into the sciatic nerve, or pharyngeal, superior, and recurrent laryngeal nerves among several other merge into the vagus nerve); and conversely separate branches are distal to the stimulation. Although the sensing contacts are usually placed distal to the stimulation site, it is also possible to sense proximal to the stimulation site. It is further noted that dorsal roots are somatotopically organized, corresponding to specific peripheral nerves and receptive fields. A particular dorsal root branch refers to a dorsal branch on which one would expect a signal when a specific peripheral nerve is stimulated. When the specific peripheral nerve is stimulated in the same manner, no such signal would be expected on other dorsal roots due to the same stimulation on the specific nerve. Sensed signals may be related to the presence or absence of pain, and/or may be related to a patient rating for the stimulation.

FIG. 10 illustrates, by way of example and not limitation, an implantable device 1034 with at least a first cuff for use to provide neuromodulation and a second cuff for use to sense evoked potentials. In the illustrated embodiment, a lead 1037 with a connected helical coil 1038 may be used with electrodes 1039 thereon for use to deliver neural stimulation to a nerve 1042. Individual ones of the stimulation contacts may be selected to provide the modulation filed over some of the fibers on the nerve but not others. A separate sensing lead 1040 may be used to place a connected helical coil 1044 with sensing contacts 1045 thereon. Individual ones of sensing contacts 1045 may be selected to sense neural activity on some of the fibers on the nerve. Sensed signals may be related to the presence or absence of pain, and/or may be related to a patient rating for the stimulation.

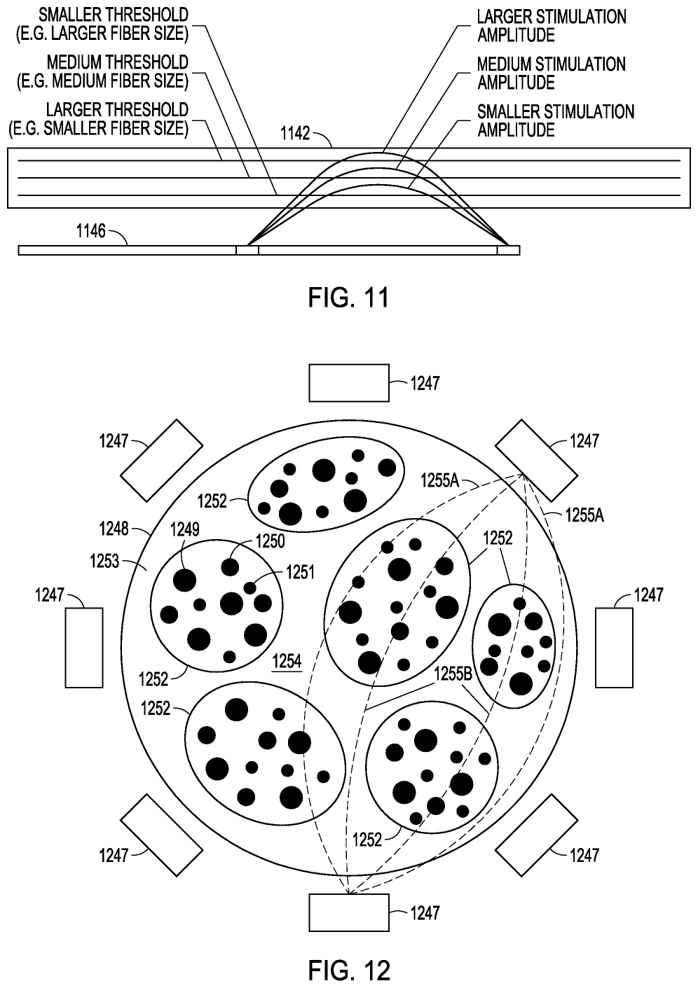

FIG. 11 is a simple illustration of different stimulation thresholds for different fiber types in a complex nerve. Peripheral nerves may have many neural pathways that are recruited at different stimulation thresholds. The intensity of the neural stimulation 1 can be adjusted by adjusting parameter(s) of the stimulation signal. For example, the amplitude of the signal (e.g., current or voltage) can be increased to increase the intensity of the signal. Other stimulation parameter(s) can be adjusted as an alternative to or in addition to amplitude. For example, stimulation intensity can vary with the frequency of the stimulation signal, a stimulation burst frequency, a pulse width and/or a duty cycle. Because different fibers have different functions and different thresholds, different neural stimulation intensities may elicit different physiological responses. Stated another way, increasing neural stimulation may trigger response “A” after reaching a certain level of intensity (e.g. a stimulation threshold), may trigger response “B” along with response “A” after reaching a higher intensity (e.g. a higher stimulation threshold), and may response “C” along with responses “A” and “B” after reaching an even higher intensity (e.g. an event higher stimulation threshold).

By way of example and not limitation, the illustration includes a bipolar stimulation lead 1146 configured to deliver nerve stimulation to the nerve. The concept illustrated in these figures may apply to other types of stimulation such unipolar stimulation and multipolar stimulation. The figures provide a simple illustration of a nerve showing, by way of a simple example, three stimulation thresholds identified as a smaller threshold, a medium threshold and a larger threshold. The threshold for a given fiber is dependent on its fiber type (size and whether nerve is myelinated) as well as its location to the stimulation electrodes. However, the concept may be simply illustrated based on fiber size (e.g., larger-sized A type fibers have a smaller stimulation threshold, medium-sized B type fibers have a medium stimulation threshold, and smaller-sized C type fibers have a larger stimulation threshold). Thus, assuming that the distance from the electrodes to the fibers are the same and assuming that an amplitude is increasing, the larger sized fibers (e.g., A type) with the lower threshold at a lower stimulation amplitude, then the medium sized fibers (e.g., B type) with the medium threshold at the medium stimulation amplitude, and then the smaller sized fibers (e.g., C type) with the large threshold at the larger stimulation amplitude.

FIG. 12 illustrates, by way of example and not limitation, stimulation or sensing electrodes around a peripheral nerve and the capability to either stimulate or sense neural activity in some fibers and not other fibers. The illustrated electrodes 1247 around the peripheral nerve 1248 may be on a nerve cuff, helical-shaped connector, wrap, and the like. The number of electrodes may vary. Furthermore, the illustration only shows electrodes in two dimensions. Those of ordinary skill in the art will understand that the electrodes are in three-dimensional space, and thus may create different stimulation and sensing vectors (e.g., into and out of the page). The individual nerve fibers, including different fiber types (e.g., illustrated as different-sized fibers 1249, 1250, and 1251), within the peripheral nerve may be grouped together in fascicles 1252, and the peripheral nerve 1248 may include many fascicles surround and separated by epineurium. External epineurium 1253 is relatively tough and surrounds the nerve, and internal epineurium 1254 is relatively loose and separates fascicles. The fascicular organization is constantly redistributed along a peripheral nerve, as interconnections are provided via interfascicular plexuses. Some embodiments may use stimulation electrodes 1247 that surround the nerve. The stimulation electrodes 1247 are capable of being individually selected to provide different modulation fields in the nerve to capture different fibers within the nerve. Furthermore, different modulation parameters (e.g., amplitude) may be used to change the shape of modulation field. For example, the size of the modulation field 1255A for a larger stimulation amplitude may be larger than the size of the modulation field 1255B for a smaller stimulation amplitude. Thus, different axons can be captured by changing the shape of the modulation field using different modulation parameters and using different stimulation electrodes. The illustration generally shows bipolar stimulation between two electrodes. Monopolar stimulation, where an indifferent electrode such as an electrode on the housing of the implantable device, may be used as well as multipolar stimulation may be used. The energy contributions of individual electrodes may be controlled, such as through the use of multiple independent current control technology which provides each electrode with its own dedicated current source capable of being controlled to provide a positive or negative polarity, such that any of the electrodes can be controlled to function as an anodic or to function as a cathodic contact. Furthermore, the system may be able to fractionalize the electrode contributions so that one or more electrodes provide the anodic contribution and one or more electrodes provide the cathodic contributions. Thus, the system is capable of producing modulation fields with various shapes and sizes to capture various groups of fibers within the peripheral nerve. Similarly, sensing electrodes that surround the nerve can be individually selected to provide various sensing vectors between or among the selected electrodes. Thus, the neural activity within different fibers within different portions of the nerve may be sensed.

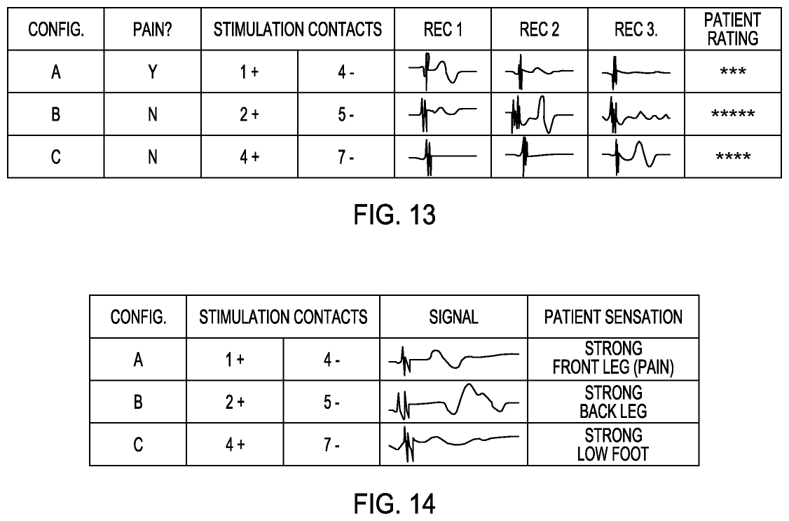

FIG. 13 illustrates, by way of example and not limitation, signals from different nerve branches which may be displayed on a graphical user interface. By way of example, the interface may display and/or solicit patient rating, patient pain rating, paresthesia vs. pain locations, and/or other patient-specific input. Different configurations may be tested, where each configuration may use different stimulation electrode combinations. For example, Configuration A may correspond to delivering stimulation using electrode 1 as an anodic electrode and electrode 4 as a cathodic electrode; Configuration B may correspond to delivering stimulation using electrode 2 as an anodic electrode and electrode 5 as a cathodic electrode, and Configuration C may correspond to delivering stimulation using electrode 4 as an anodic electrode and electrode 7 as a cathodic electrode. The stimulation-evoked signals (e.g., Recording 1, Recording 2, Recording 3) may be collected from distinct branches, and these recordings for each configuration may be displayed along with sensory input from patient. For example, the patient may provide an indication of pain for each configuration, and may rate the therapeutic effects for the neuromodulation delivered in each configuration. Additional information may be displayed such as stimulation parameters (e.g., amplitude, pulse width) used for each configuration.

FIG. 14 illustrates, by way of example and not limitation, signals from the same nerve branches which may be displayed on a graphical user interface. Thus, rather than sensing different nerve branches, the stimulation-evoked signals may be collected from different sides of same nerve. The illustrated configurations are the same as was illustrated in FIG. 13. The displayed recording may be combined with sensory input from patient. The interface, for example, may display and/or solicit patient rating, patient pain rating, paresthesia vs. pain locations, and/or other patient-specific input. In the illustrated embodiment, the patient provide input on patient sensation for each configuration, where the patient feels strong paresthesia in the front leg and still feels pain for Configuration A, where the patient feels strong paresthesia in the back of a leg without pain for Configuration B, and where the patient feels strong paresthesia in the low foot without pain for Configuration C.

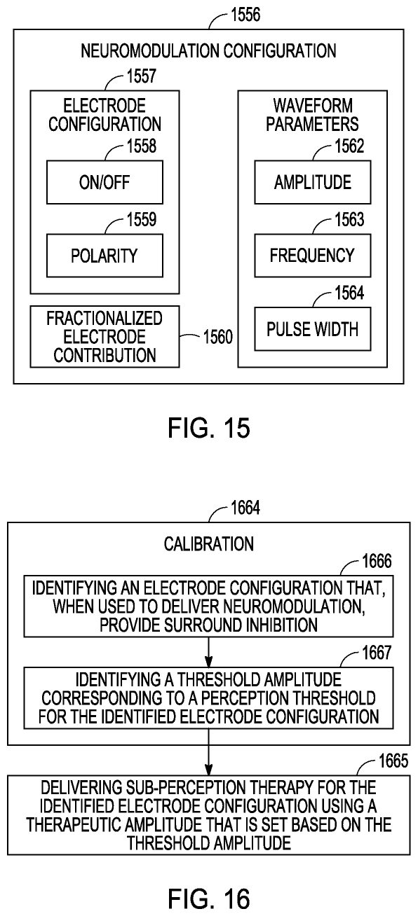

FIG. 15 illustrates, by way of example and not limitation, some neuromodulation parameters that may contribute to a neuromodulation configuration. Any of these neuromodulation parameters may be displayed for each of the configurations (see FIGS. 13 and 14). For example, the neuromodulation configuration 1556 includes the electrode configuration 1557, which includes whether the electrode is contributing energy to the modulation field (e.g., ON/OFF) 1558, and the polarity of the electrode 1559. The neuromodulation configuration 1556 may also include the fractionalized electrode contributions 1560 of each electrode used to contribute energy to the modulation field. The contributions of each anodic electrode add up to 100% of the total anodic contribution, and the contributions of each cathodic electrode add up to 100% of the total cathodic contribution. Furthermore, the neuromodulation configuration 1556 may include waveform parameters 1561. For example, waveform parameters 1561 for a pulse generator that is configured to generate pulses may include an amplitude 1562, a frequency 1563 and a pulse width 1564. More complicated waveforms may be used, such as different shaped pulses, different pulse patterns including different regular and irregular patterns of pulses. Of note, stimulation waveforms may be static (using constant stimulation parameters such as a constant pulse amplitude, constant pulse width or constant pulse frequency) or dynamic meaning any or a combination of its parameters are constantly changing following any desired mathematical function. For example, a stimulation waveform may have a current or voltage amplitude that is constantly changing according to a sinusoidal signal, or according to a random Poisson model. In another example, a stimulation waveform may have a frequency parameter constantly changing according to a sinewave signal, or a random signal, or exponential signal. Any parameter, such as but not limited to pulse width, charge per second, charge per phase, duty cycle, and the like, can be modulated with a mathematical construct to create a time varying signal. This has potential advantages to prevent habituation in the therapeutic response, as it is harder to adapt to a constantly changing stimulation; and also has the advantage of enabling broader coverage of the targeted anatomical region (surround inhibitory field or other) based on internal studies conducted in humans [e.g., see https://clinicaltrials.gov/ct2/show/NCT02988713?term=NCT02988713&draw=2&rank=1]. Dynamic waveform patterns may change in time and/or space (e.g., changes in the active electrodes used for stimulation and the fractionalized contribution of each active electrode). Thus, by way of example and not limitation, a dynamic pattern may change one or more of frequency, pulse width, amplitude, duty cycle, charge per second, charge per phase, fractionalizations, and spatial electrodes used to deliver the stimulation.

FIG. 16 illustrates, by way of example and not limitation, a method for calibrating and delivering a sub-perception therapy using peripheral neuromodulation. The method may be performed using a plurality of neuromodulation electrode contacts configured and arranged for use in delivering neuromodulation to a target peripheral nerve. As identified above, the target peripheral nerve includes a plurality of fibers, and the plurality of neuromodulation electrode contacts is configurable into a plurality of electrode configurations for stimulating different subsets of fibers within the plurality of fibers. The illustrated process involves a method for calibrating the therapy 1665, and delivering the therapy 1666. Calibrating 1665 may include: identifying an electrode configuration that, when used to deliver the neuromodulation, stimulates an inhibitory surround receptive field 1667, and identifying a threshold amplitude corresponding to a perception threshold for the identified electrode configuration 1668. It is noted that the threshold amplitude determined at 1667 may correspond to an evoked neural response, such as an ECAP, that is detectable by a nerve sensor in addition or alternatively to corresponding to a perception threshold. At 1665, a sub-perception therapy for the identified electrode configuration may be delivered using a therapeutic amplitude that is set based on the threshold amplitude. For example, the therapeutic amplitude may be set as a percentage of the threshold amplitude (e.g., 40% to 90% of the perception threshold).

Identifying the electrode configuration may include delivering neuromodulation energy in a process to identify a neuromodulation configuration that stimulates the inhibitory surround receptive field around the localized pain region, where the identified neuromodulation configuration includes: waveform parameters, and the identified electrode configuration. For example, the method may include independently controlling the current to each of the plurality of neuromodulation electrode contacts to control fractionalized current contributions to individual electrode contacts within the identified electrode configuration. Identifying the electrode configuration may include using sensing electrode contacts to sense evoked neural responses, where the sensing electrode contacts are configurable into a plurality of sensing configurations for sensing evoked neural responses in different subsets of fibers within the plurality of fibers, and recording data corresponding to the received electrical signal. The identifying a threshold amplitude may include performing a threshold process, which may include stepping up an adjustable amplitude of the neuromodulation until a patient perceives paresthesia, stepping up the adjustable amplitude until a neural response is evoked or suppressed, stepping up the adjustable amplitude until a muscle twitch or an EMG signal is evoked or suppressed, stepping down the adjustable amplitude of the neuromodulation until the patient fails to perceive the paresthesia, stepping down the adjustable amplitude until the neural response is evoked or suppressed, or stepping down the adjustable amplitude until a muscle twitch or an EMG signal is evoked or suppressed. It is noted that some nerves, such as but not limited to the sciatic and vagus nerves, are multimodal nerves that contain sensory, motor and other functional nerve fibers. Motor fibers (especially large, fast motor fibers or large, fast proprioceptive fibers that produce motor reflexes) are relatively low threshold. Therefore, muscle twitch or an electromyogram may provide an indication of a threshold.

FIG. 17 illustrates, by way of example and not limitation, a method for calibrating the peripheral neuromodulation for surround receptive field(s) and/or center receptive field(s) using patient inputs and/or sensed evoked neural responses. At 1768 the process includes testing neuromodulation configurations. The tested configurations may be super-perception configurations. The system may receive inputs from a user, such as the patient or clinician. The inputs may include perception (or paresthesia) and pain. The system may also detect evoked neural responses on branches 1770. Some embodiments may identify or save the configuration for a center received field 1771 and some embodiments may identify/save configurations for surround receptive field 1772. This may be performed by implementing an algorithm to distinguish between a center receptive field or a surround receptive field based on a relation among patient sensation, pain and the evoked neural response including features in the evoked neural response. The algorithm may be configured to infer from an overlap in the patient sensation and the pain and from features the evoked neural response that the center receptive field is stimulated or that the surround receptive field is stimulated via low-threshold fibers (See Configuration A above). Rather than relying on perception, it is noted that the electrode configuration may be identified by applying neuromodulation, receiving patient input regarding pain, receiving input (e.g., sensor input) regarding evoked neural response in fibers of the peripheral nerve, and implementing an algorithm to distinguish between stimulation of fibers from the center receptive field or the inhibitory surround receptive field based the patient input and the evoked neural response. Some embodiments may use both sensor inputs regarding evoked neural responses and user inputs regarding patient perception. The algorithm may be configured to infer from slight discordance among the patient sensation and the pain, and from slight distinctions in the evoked neural response that the surround receptive field is stimulated (see Configuration B above). The algorithm may be configured to infer from at least one of a strong sensation or a strong distinction in the evoked neural response that the surround receptive field is stimulated or a different receptive field is stimulated (see Configuration C above). The algorithm may relate patient sensation and pain ratings to which branch(es) or contact(s) on which an evoked neural response was detected to infer center or surround.

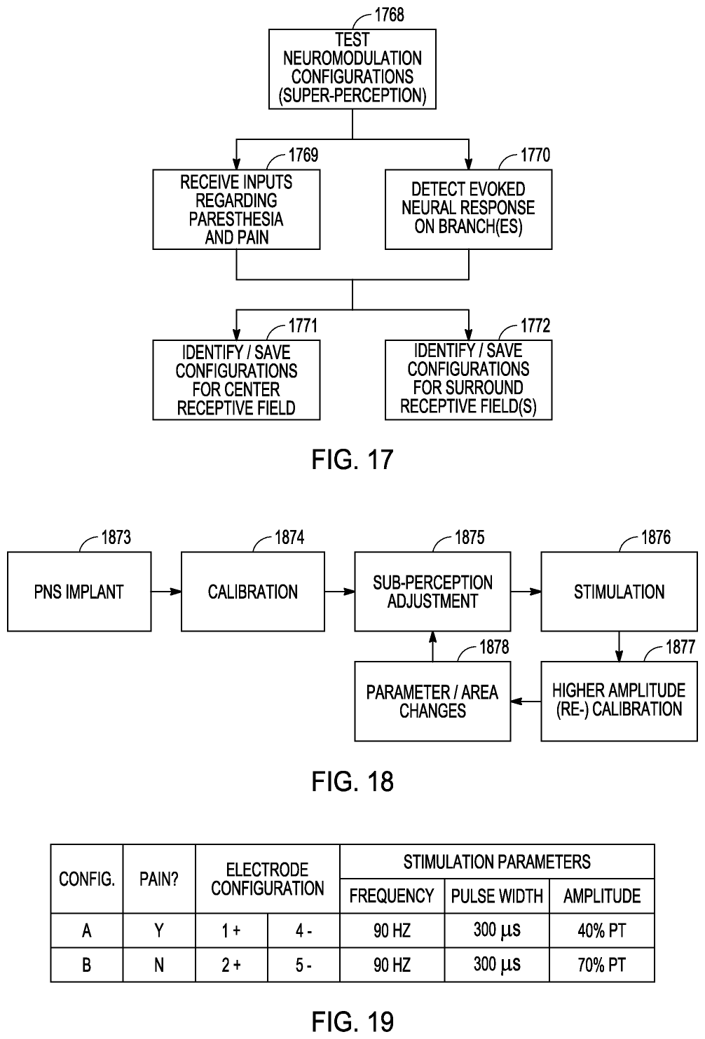

FIG. 18 illustrates, by way of example and not limitation, a general workflow for closed-loop control of a neuromodulation therapy. The workflow may begin with implanting one or more peripheral nerve stimulation (PNS) devices at 1873. As illustrated at 1874, the PNS device may be calibrated to deliver the appropriate neuromodulation parameters (e.g., electrode configuration, fractionalization, etc.) to provide the desired surround receptive field (RF). The workflow may then adjust the neuromodulation parameters based on the calibration parameters to provide sub-perception neuromodulation parameters 1875. For example, the amplitude may be set to 40% to 70% of the perception threshold amplitude. At 1876, the sub-perception stimulation may be delivered using the PNS device(s). As identified earlier, the system may be configured with timing channels that correspond to different groups of electrodes; and given the independent control of stimulation to electrodes in channels, each of the channels may correspond to an “area” of stimulation. Various embodiments may vary stimulation amplitudes for multiple channels/“areas” that correspond to the user-defined “center” or “surround” designation of a given branch or signal cluster (e.g., “surround” stimulation is applied at a higher amplitude than “center’ stimulation). The stimulation delivered for the therapy is sub-perception. Some embodiments may be configured to calibrate or recalibrate by stimulating at higher amplitudes, as illustrated at 1877, such that the patient can perceive the stimulation, to periodically verify the peripherally evoked signals. The verification process that delivers the higher amplitude signal may be periodically performed or performed based on user input such as if the patient reports a change in therapy. The calibration process may result in change(s) to the neuromodulation configuration, such as changes to parameters such as amplitude and changes to electrode combinations (e.g., changes to areas) 1878. Adjustments may be made to these changes to provide sub-perception neuromodulation parameters 1875, and the sub-perception therapy may be delivered at 1876.

FIG. 19 illustrates, by way of example and not limitation, programming configurations A and B for providing neuromodulation to two different areas. These configurations may be selected based on the processes illustrated in FIGS. 13 and 14. Configuration A may be selected as the stimulation covers the pain (center stimulation), and configuration B may be selected as providing a strong surround stimulation, based at least in part on the patient rating (see FIG. 13). The amplitude for the center stimulation may be delivered at 40% of the perception threshold amplitude, and the amplitude for the surround stimulation may be delivered at 70% of the perception threshold. Perception threshold (PT) weightings may be pre-set or adjustable by a programmer/the patient. Stimulation could be defined in raw mA and/or relative to an electrophysiological (EP) threshold such as an evoked neural response, perception threshold (PT) such as a threshold for sensing paresthesia, or another metric.

Peripheral nerve stimulation may be viewed as a less invasive (and, therefore, more qualitatively acceptable) therapy by patients, and effects of PNS may be similar to those of SCS. The PNS device may be full implantable, including the electrodes, the lead(s), the waveform (e.g., pulse) generator, the controller and the power supply. Some embodiments are partially implanted and partially external. Examples are provided using some of the components previously illustrated in FIGS. 2 and 4. Sensors are not illustrated, by may be implantable or external.

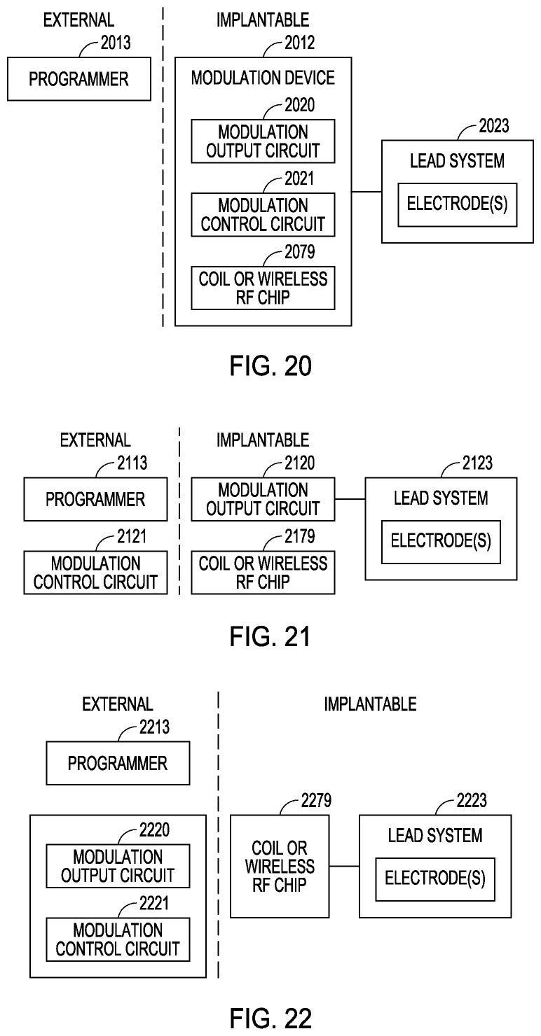

FIG. 20 illustrates, by way of example and not limitation, a fully implantable PNS device and an external programmer. An external programmer 2013 is configured to communicate with the implantable modulation device 2012, which is connected to the implantable lead system 2023, which may include neuromodulation electrode contacts on at least one cuff, an anchored patch, or a needle array. The modulation device 2012 may include the modulation output circuit 2020, modulation control circuit, and a coil 2079 or wireless RF chip that may be used to wirelessly communicate with the external programmer 2013. The implantable modulation output circuit 2020 may be connected to the lead system 2023 to deliver the peripheral nerve stimulation. The wireless communication may be RF or inductive communication. In some embodiments, the PNS device may be configured to wireless receive power from the programmer 2013 or another external device. The received power may be used to recharge a battery in the modulation device 2012 or to directly power the modulation device using the coil 2079 or another coil.

FIG. 21 illustrates, by way of example and not limitation, a partially implantable PNS device. An external programmer 2013 may be configured to communication with an external modulation control circuit 2121, either using a wireless or wired connection. The modulation control circuit 2121 may be configured to control the implantable modulation output circuit 2120 using a wireless signal. The implantable modulation output circuit 2120 may be connected to the lead system 2123 to deliver the peripheral nerve stimulation. The lead system 2123 may include neuromodulation electrode contacts on at least one cuff, an anchored patch, or a needle array. The wireless communication may be RF or inductive communication via the implantable coil or wireless RF chip 2179. In some embodiments, the PNS device may be configured to wireless receive power from the programmer 2113 or another external device. The received power may be used to recharge an implantable battery used to power the delivery of the neuromodulation, or to directly power the delivery of the neuromodulation using the coil 2179 or another coil.

FIG. 22 illustrates, by way of example and not limitation, a partially implantable PNS device. An external programmer 2213 may be configured to communication with an external device or system which includes an external modulation control circuit 2221 and a modulation control circuit 2222, either using a wireless or wired connection. The modulation control circuit 2221 may be configured to control the implantable modulation output circuit 2220 using a wired or a wireless signal. The external modulation output circuit 2220 may be configured to control the delivery of energy to the lead system to deliver the peripheral nerve stimulation. The implantable portions of the PNS device may be configured to wireless receive power from the programmer 2213 or another external device. The received power may be used to recharge an implantable battery used to power the delivery of the neuromodulation, or to directly power the delivery of the neuromodulation using the coil 2279 or another coil.

Some embodiments may be entirely externalized, such as a patient wearable device positioned around the target region if the target region can be accessed using the external device. For example, an extremity band with electrodes may be placed around legs or arms, and can be made with stretchable material or with an adjustable diameter but fixed electrodes positions. Some embodiments may include a patch or a needle array. The electrode contacts in the patch or needle array may press against the skin without breaking the skin. In some embodiments, the electrodes contact may be transcutaneously inserted to a subcutaneous target. The external device may be within one package, such as a watch, or may have more than one component operably connected to provide communication and/or power via wired or wireless (RF or inductive) technologies.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method performed using a plurality of neuromodulation electrode contacts configured and arranged for use in delivering neuromodulation to a target peripheral nerve, wherein the target peripheral nerve includes a plurality of fibers, and the plurality of neuromodulation electrode contacts is configurable into a plurality of electrode configurations for stimulating different subsets of fibers within the plurality of fibers, the method comprising:
identifying an electrode configuration that, when used to deliver the neuromodulation, stimulates fibers from an inhibitory surround receptive field;
identifying a threshold amplitude corresponding to a perception threshold, a motor or an EMG threshold, or an evoked neural threshold for the identified electrode configuration; and
delivering sub-perception therapy for the identified electrode configuration using a therapeutic amplitude that is set based on the threshold amplitude.

2. The method of claim 1, wherein the identifying the electrode configuration includes delivering neuromodulation energy in a process to identify a neuromodulation configuration that stimulates the fibers from the inhibitory surround receptive field around the localized pain region, wherein the identified neuromodulation configuration includes: waveform parameters, and the identified electrode configuration.

3. The method of claim 2, further comprising independently controlling the current to each of the plurality of neuromodulation electrode contacts to control fractionalized current contributions to individual electrode contacts within the identified electrode configuration.

4. The method of claim 1, wherein the identifying a threshold amplitude includes performing a threshold process, wherein the threshold process includes:
stepping up an adjustable amplitude of the neuromodulation until a patient perceives paresthesia;
stepping up the adjustable amplitude until a neural response is evoked or suppressed;
stepping up the adjustable amplitude until a muscle twitch or an EMG signal is evoked or suppressed;
stepping down the adjustable amplitude of the neuromodulation until the patient fails to perceive the paresthesia;
stepping down the adjustable amplitude until the neural response is evoked or suppressed; or
stepping down the adjustable amplitude until the muscle twitch or the EMG signal is evoked or suppressed.

5. The method of claim 1, wherein the identifying the electrode configuration includes:
using sensing electrode contacts to sense evoked neural responses, wherein the sensing electrode contacts are configurable into a plurality of sensing configurations for sensing evoked neural responses in different subsets of fibers within the plurality of fibers, and
recording data corresponding to the received electrical signal.

6. The method of claim 5, wherein the sensing electrode contacts are configured for sensing evoked neural responses on different sides of the peripheral nerve or distinct branches of the peripheral nerve, method further comprising:
receiving a sensory input from a patient; and
displaying the sensed evoked responses and the received sensory input on a user interface.

7. The method of claim 6, wherein the received sensory input from the patient includes at least one of pain, patient sensation or patient rating corresponding to test neuromodulation configurations.

8. The method of claim 7, further comprising displaying the electrode configuration corresponding to the test neuromodulation configurations.

9. The method of claim 1, wherein the identifying the electrode configuration includes:
applying super-perception neuromodulation;
receiving patient input regarding paresthesia and pain;
receiving input regarding evoked neural response in fibers of the peripheral nerve; and
implementing an algorithm to distinguish between stimulation of fibers from the center receptive field or the inhibitory surround receptive field based on a relation among patient sensation, pain, and the evoked neural response including features in the evoked neural response.

10. The method of claim 9, wherein the algorithm is configured to infer from an overlap in the patient sensation and the pain and from features the evoked neural response that low-threshold fibers from the center receptive field or the inhibitory surround receptive field is stimulated.

11. The method of claim 9, wherein the algorithm is configured to infer from slight discordance among the patient sensation and the pain, and from slight distinctions in the evoked neural response that the fibers from the inhibitory surround receptive field is stimulated.

12. The method of claim 9, wherein the algorithm is configured to infer from at least one of a strong sensation or a strong distinction in the evoked neural response that the inhibitory surround receptive field or a different receptive field is stimulated.

13. The method of claim 1, wherein the therapeutic amplitude is less than the threshold amplitude and is set as a percentage of the threshold amplitude.

14. The method of claim 1, wherein the neuromodulation configuration includes a pulse frequency within a range of 50 Hz to 100 Hz and a pulse width within a range of 210-230 μs.

15. The method of claim 14, wherein the pulse frequency is 90 Hz.

16. The method of claim 1, wherein the identifying the electrode configuration includes:
applying neuromodulation;
receiving patient input regarding pain;
receiving input regarding a motor or electromyogram response or an evoked neural response in fibers of the peripheral nerve; and
implementing an algorithm to distinguish between stimulation of fibers from the center receptive field or the inhibitory surround receptive field based the patient input and the evoked neural response.

17. A non-transitory machine-readable medium including instructions, which when executed by a machine, cause the machine to perform a method using a plurality of neuromodulation electrode contacts configured and arranged for use in delivering neuromodulation to a target peripheral nerve, wherein the target peripheral nerve includes a plurality of fibers, and the plurality of neuromodulation electrode contacts is configurable into a plurality of electrode configurations for stimulating different subsets of fibers within the plurality of fibers, the method comprising:
identifying an electrode configuration that, when used to deliver the neuromodulation, stimulates fibers from an inhibitory surround receptive field;
identifying a threshold amplitude corresponding to a perception threshold, a motor or an EMG threshold, or an evoked neural threshold for the identified electrode configuration; and
delivering sub-perception therapy for the identified electrode configuration using a therapeutic amplitude that is set based on the threshold amplitude.

18. The non-transitory machine-readable medium of claim 17, wherein the identifying the electrode configuration includes delivering neuromodulation energy in a process to identify a neuromodulation configuration that stimulates the inhibitory surround receptive field around the localized pain region, wherein the identified neuromodulation configuration includes the identified electrode configuration from the plurality of electrode configurations.

19. The non-transitory machine-readable medium of claim 17, wherein the therapeutic amplitude is less than the threshold amplitude and is set as a percentage of the threshold amplitude.

20. A system, comprising:
a plurality of neuromodulation electrode contacts configured and arranged for use in delivering neuromodulation to a target peripheral nerve, wherein the target peripheral nerve includes a plurality of fibers, and the plurality of neuromodulation electrode contacts is configurable into a plurality of electrode configurations for stimulating different subsets of fibers within the plurality of fibers;
a waveform generator configured for use to generate neuromodulation energy; and
a controller configured for use for:
identifying an electrode configuration that, when used to deliver the neuromodulation, stimulates fibers from an inhibitory surround receptive field;
identifying a threshold amplitude corresponding to either a perception threshold, a motor or an electromyogram (EMG) threshold, or an evoked neural threshold for the identified electrode configuration; and
delivering sub-perception therapy for the identified electrode configuration using a therapeutic amplitude that is set based on the threshold amplitude.
